(19) United States
(12) Patent Application Publication
(10) Pub. No.: US 2022/0047701 A1
(43) Pub. Date: Feb. 17, 2022

(54) COMBINATION OF HER2/NEU ANTIBODY WITH HEME FOR TREATING CANCER

(71) Applicants: INSERM (Institut National de la Santé et de la Recherche Médicale), Paris (FR); Université de Paris, Paris (FR); Sorbonne Université, Paris (FR)

(72) Inventors: Jordan DIMITROV, Paris (FR); Sébastien LACROIX-DESMAZES, Paris (FR); Annaelle MAREY JAROSSAY, Paris (FR)

(21) Appl. No.: 17/274,592

(22) PCT Filed: Sep. 9, 2019
(86) PCT No.: PCT/EP2019/073938

§ 371 Date: Mar. 9, 2021

(30) Foreign Application Priority Data
Sep. 10, 2018 (EP) 18306187.8

(51) Int. Cl. A61K 39/395; A61K 31/555; C07K 14/725; C07K 16/32; C07F 15/02; A61P 35/00

(52) U.S. Cl. CPC A61K 39/3955; A61K 31/555; C07K 14/7051; C07K 2317/92; C07F 15/025; A61P 35/00; C07K 2317/622; C07K 16/32

(57) ABSTRACT

The present invention relates to a method of treating HER2/NEU overexpressing cancers. The inventors discovered that the heme-mediated formation of dimers and in general oligomers of Trastuzumab is associated with an improved complement-mediated cytotoxicity on breast cancer cells. The present data highlight that the sensitivity to heme of Trastuzumab, may have major repercussion on its therapeutic activity. Thus the invention relates to the combination of an HER2/neu antibody with a heme and/or of its oligomers and its therapeutic composition in the HER2/NEU characteristic cancer treatment.

### FIELD OF THE INVENTION

The present invention relates to a combination of an HER2/neu antibody with a heme and its application.

### BACKGROUND OF THE INVENTION

Antibodies (Abs) contribute to the immune defence by highly specific recognition of antigenic determinants displayed by pathogens. Besides Abs that interact with conventional antigens i.e. proteins, carbohydrates or lipids, normal immune repertoires contain a fraction of immunoglobulins that can bind to different low molecular weight organic compounds. These organic compounds include some essential for the aerobic metabolism molecules, such as flavin-containing cofactors [1, 2, 3], adenosine triphosphate [4] and heme (iron protoporphyrin IX) [5]. The origin and biological significance of these Abs, however, remain poorly understood. The interaction of some compounds with Abs results in considerable functional consequences. Thus, exposure to heme is known to induce appearance of reactivity of Abs to previously not recognized protein or lipid antigens [6, 7, 8, 9, and 10]. Heme-exposed Abs bound their new target antigens with values of the equilibrium dissociation constant (KD) in low nanomolar range [9, 11]. Importantly, the acquisition of new antigen-binding specificities correlates with an acquisition of a potential to neutralize pathogens and with a substantial increase in the anti-inflammatory activity of the immunoglobulins, suggesting that the cofactor-bound Abs might have physiological relevance [12-14]. It is noteworthy that under physiological conditions heme can be found exclusively intracellularly, predominantly bound to different proteins (hemoproteins). However, in cases of an extensive tissue damage or hemolysis large quantities of heme can be liberated in the extracellular compartment and potentially interacts with circulating immunoglobulins [15-17].

The use of monoclonal therapeutic Abs in therapy of cancer has successfully transformed the treatment strategies over the past 20 years [18]. One of the essential characteristics that allows rapid and targeted action of the therapeutic Abs is their high specificity. McIntyre et al. have demonstrated that some of the therapeutic monoclonal Abs that are currently used in the clinical practice can acquire a strong autoreactivity upon in vitro contact with heme [19]. This finding suggest that clinically approved Abs with stringently validated target specificity can also interact with low molecular weight substance and experience functional alterations upon these interactions. This observation could be especially important in case of cancer therapy where the massive cellular death can result in the release of various intracellular components and therefore present an environment, which could modify the binding specificity and the functional properties of the therapeutic Abs [20]. Nevertheless, the impact of heme binding on functional activity of the therapeutic monoclonal Abs has never been investigated.

### SUMMARY OF THE INVENTION

The current study sought to understand the molecular and functional consequences of interaction of Trastuzumab with heme. Trastuzumab and its analogues are human epidermal factor receptor (HER2/neu)-specific humanized IgG1 that has been mainly used for the treatment of ERBB2 overexpressing forms of breast cancer. HER2 possesses, among all HER family proteins, the strongest catalytic kinase activity and functions as the most active signalling complex after dimerization. Overexpression of HER2 in several malignancies lead to an increased dimerization which initiates a strong pro-tumorigenic signalling cascade. Surprisingly it was demonstrated in this study that heme binds with a high affinity to variable region of Trastuzumab. This interaction, which results in a Fab-dependent self-association of Trastuzumab however, do not perturb the binding of the antibody to its cognate antigen. In contrary, the heme-mediated formation of dimers and in general oligomers of Trastuzumab was found to be associated with an improved complement-mediated cytotoxicity on breast cancer cells. The present data highlight that the sensitivity to heme of Trastuzumab, may have major repercussion on its therapeutic activity. The heme-mediated dimerization of therapeutic antibodies may represent an innovative strategy for improvement of therapeutic effect of antibodies.

Thus, the present invention relates to a combination of an HER2/neu antibody with a heme. Particularly, the invention is described by the claims.

### DETAILED DESCRIPTION OF THE INVENTION

A first aspect of the invention relates to a combination of an HER2/neu antibody with a heme.

In a particular embodiment, the invention relates to a combination of an HER2/neu specific therapeutic antibody with a heme.

As used herein the term “HER2/neu” also known as receptor tyrosine-protein kinase erbB-2, CD340 (cluster of differentiation 340), proto-oncogene Neu, Erbb2 (rodent), or ERBB2 (human), frequently called HER2 HER2/neu is a 185 KDa protein with homology to epidermal growth factor receptor (EGFR). Along with HER3 (ErbB3) and HER4 (ErbB4), these proteins constitute the type 1 growth receptor gene family. Amplification or over-expression of this oncogene has been shown to play an important role in the development and progression of certain aggressive types of cancer.

As used herein the term “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs.

In one embodiment, the antibody of the invention is an antigen biding fragment selected from the group consisting of a Fab, a F(ab)′2, a single domain antibody, a ScFv, a Sc(Fv)2, a diabody, a triabody, a tetrabody, an unibody, a minibody, a maxibody, a small modular immunopharmaceutical (SMIP), minimal recognition units consisting of the amino acid residues that mimic the hypervariable region of an antibody as an isolated complementary determining region (CDR), and fragments which comprise or consist of the VL or VH chains.

The term “antigen binding fragment” of an antibody, as used herein, refers to one or more fragments of an intact antibody that retain the ability to specifically binds to a given antigen (e.g., HER2/neu). Antigen biding functions of an antibody can be performed by fragments of an intact antibody. Examples of biding fragments encompassed within the term antigen biding fragment of an antibody include a Fab fragment, a monovalent fragment consisting of the VL, VH, CL and CH1 domains; a Fab′ fragment, a monovalent fragment consisting of the VL, VH, CL, CH1 domains and hinge region; a F(ab′)2 fragment, a bivalent fragment comprising two Fab′ fragments linked by a disulfide bridge at the hinge region; an Fd fragment consisting of VH domains of a single arm of an antibody; a single domain antibody (sdAb) fragment (Ward et al., 1989 Nature 341:544-546), which consists of a VH domain or a VL domain; and an isolated complementary determining region (CDR). Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (ScFv); see, e.g., Bird et al., 1989 Science 242:423-426; and Huston et al., 1988 proc. Natl. Acad. Sci. 85:5879-5883). “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2. Such single chain antibodies include one or more antigen biding portions or fragments of an antibody. These antibody fragments are obtained using conventional techniques known to those skilled in the art, and the fragments are screened for utility in the same manner as are intact antibodies. A unibody is another type of antibody fragment lacking the hinge region of IgG4 antibodies. The deletion of the hinge region results in a molecule that is essentially half the size of traditional IgG4 antibodies and has a univalent binding region rather than the bivalent biding region of IgG4 antibodies. Antigen binding fragments can be incorporated into single domain antibodies, SMIP, maxibodies, minibodies, intrabodies, diabodies, triabodies and tetrabodies (see, e.g., Hollinger and Hudson, 2005, Nature Biotechnology, 23, 9, 1126-1136). The term “diabodies” “tribodies” or “tetrabodies” refers to small antibody fragments with multivalent antigen-binding sites (2, 3 or four), which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Antigen biding fragments can be incorporated into single chain molecules comprising a pair of tandem Fv segments (VH-CH1-VH-CH1) Which, together with complementary light chain polypeptides, form a pair of antigen binding regions (Zapata et al., 1995 Protein Eng. 8 (10); 1057-1062 and U.S. Pat. No. 5,641,870).

The Fab of the present invention can be obtained by treating an antibody which specifically reacts with HER2/neu with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained treating an antibody which specifically reacts with HER2/neu with a protease, pepsin. Also, the F(ab′)2 can be produced by binding Fab′ described below via a thioether bond or a disulfide bond.

The Fab′ of the present invention can be obtained treating F(ab′)2 which specifically reacts with HER2/neu with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well known technology called CDR grafting may be used, which involves selecting the complementary determining regions (CDRs) from a donor scFv fragment, and grafting them onto a human scFv fragment framework of known three dimensional structure (see, e. g., W098/45322; WO 87/02671; U.S. Pat. Nos. 5,859,205; 5,585,089; 4,816,567; EP0173494).

Domain Antibodies (dAbs) are the smallest functional binding units of antibodies—molecular weight approximately 13 kDa—and correspond to the variable regions of either the heavy (VH) or light (VL) chains of antibodies. Further details on domain antibodies and methods of their production are found in U.S. Pat. Nos. 6,291,158; 6,582,915; 6,593,081; 6,172,197; and 6,696,245; US 2004/0110941; EP 1433846, 0368684 and 0616640; WO 2005/035572, 2004/101790, 2004/081026, 2004/058821, 2004/003019 and 2003/002609, each of which is herein incorporated by reference in its entirety.

UniBodies are another antibody fragment technology, based upon the removal of the hinge region of IgG4 antibodies. The deletion of the hinge region results in a molecule that is essentially half the size of a traditional IgG4 antibody and has a univalent binding region rather than a bivalent binding region. Furthermore, because UniBodies are about smaller, they may show better distribution over larger solid tumors with potentially advantageous efficacy. Further details on UniBodies may be obtained by reference to WO 2007/059782, which is incorporated by reference in its entirety.

As used herein the term “HER2/neu antibody” refers to all known antibodies, which possess a negative effect on HER2/neu protein. HER2/neu antibodies include but are not limited to Trastuzumab, Pertuzumab, 2B1, Ado-Trastuzumab-Emtansine also called T-DM1 or Trastuzumab-DM1, MDX-H210, Bevacizumab and 4D5scFv-PE40 (see for example Chung A et al. 2013; Nahta R et al. 2006; Sokolova EA, 2014).

In a particular embodiment the term HER2/neu antibody refers to Trastuzumab.

As used herein the term “heme” refers to a deep red, iron-containing compound, C34H32FeN4O4, which constitutes the nonprotein component of hemoglobin and certain other proteins and may be selected in the group consisting of heme a, heme b, heme c, heme d, heme di, heme o, heme P460, siroheme, Fe porphyrines such as Fe (III) mesoporphyrin IX, Fe (III) protoporphyrin IX, Fe (III) deuteroporphyrin IX, Fe (III) hematoporphyrin IX or Fe (III) coproporphyrin (see for example J. T. Hoard, 2017). Thus according to the invention, the heme of the invention and the analogue of heme (like siroheme and porphyrines) contain iron (Fe) in their structures.

In a particular embodiment, the heme of the invention is a Fe porphyrines, and more particularly, the Fe (III) deuteroporphyrin IX.

In a particular embodiment, the heme of the invention can be activated by using carboxyl-reactive conjugation agents (1-ethyl-3-(3-dimethylaminopropyl)carbodiimide hydrochloride; 1,1′ -Carbonyldiimidazole etc).

A second aspect of the present invention also provides chimeric antigen receptors (CARs) comprising an antigen binding domain of antibody according to the invention combined with a heme according to the present invention.

As used herein, the term “chimeric antigen receptor” or “CAR” has its general meaning in the art and refers to an artificially constructed hybrid protein or polypeptide containing the antigen binding domains of an antibody (e.g., scFv) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In some embodiments, said CAR comprises at least one VH and/or VL sequence of the HER2/neu antibody of the present invention. The chimeric antigen receptor of the present invention also comprises an extracellular hinge domain, a transmembrane domain, and an intracellular T cell signaling domain.

In some embodiments, the invention provides CAR comprising an antigen-binding domain comprising, consisting of, or consisting essentially of, a single chain variable fragment (scFv) of the HER2/neu antibody. In some embodiments, the antigen binding domain comprises a linker peptide. The linker peptide may be positioned between the light chain variable region and the heavy chain variable region.

In some embodiments, the CAR comprises an extracellular hinge domain, a transmembrane domain, and an intracellular T cell signaling domain selected from the group consisting of CD28, 4-1BB, and CD3ζ intracellular domains. CD28 is a T cell marker important in T cell co-stimulation. 4-1BB transmits a potent costimulatory signal to T cells, promoting differentiation and enhancing long-term survival of T lymphocytes. CD3ζ associates with TCRs to produce a signal and contains immunoreceptor tyrosine-based activation motifs (ITAMs).

In some embodiments, the chimeric antigen receptor of the present invention can be glycosylated, amidated, carboxylated, phosphorylated, esterified, N-acylated, cyclized via, e.g., a disulfide bridge, or converted into an acid addition salt and/or optionally dimerized or polymerized.

The invention also provides a nucleic acid encoding for a chimeric antigen receptor of the present invention. In some embodiments, the nucleic acid is incorporated in a vector as such as described above.

The present invention also provides a host cell comprising a nucleic acid encoding for a chimeric antigen receptor of the present invention. While the host cell can be of any cell type, can originate from any type of tissue, and can be of any developmental stage; the host cell is a T cell, e.g. isolated from peripheral blood lymphocytes (PBL) or peripheral blood mononuclear cells (PBMC). In some embodiments, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell can be any type of T cell and can be of any developmental stage, including but not limited to, CD4+/CD8+ double positive T cells, CD4+ helper T cells, e.g., Th2 cells, CD8+ T cells (e.g., cytotoxic T cells), tumor infiltrating cells, memory T cells, naive T cells, and the like. The T cell may be a CD8+ T cell or a CD4+ T cell.

The population of those T cells prepared as described above can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg. Adoptive immunotherapy of cancer refers to a therapeutic approach in which immune cells with an antitumor reactivity are administered to a tumor-bearing host, with the aim that the cells mediate either directly or indirectly, the regression of an established tumor. Transfusion of lymphocytes, particularly T lymphocytes, falls into this category. Currently, most adoptive immunotherapies are autolymphocyte therapies (ALT) directed to treatments using the patient's own immune cells. These therapies involve processing the patient's own lymphocytes to either enhance the immune cell mediated response or to recognize specific antigens or foreign substances in the body, including the cancer cells. The treatments are accomplished by removing the patient's lymphocytes and exposing these cells in vitro to biologics and drugs to activate the immune function of the cells. Once the autologous cells are activated, these ex vivo activated cells are reinfused into the patient to enhance the immune system to treat cancer. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the T cells with the desired specificity, on the age and weight of the recipient, on the severity of the targeted condition and on the immunogenicity of the targeted Ags. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. For example, if cells that are specific for a particular Ag are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

In particular, the cells of the present invention are particularly suitable for the treatment of cancer. Accordingly, a further object of the present invention relates to a method of treating cancer in a subject in need thereof comprising administering to the subject a therapeutically effective amount of a population of cells of the present invention.

Particularly, invention relates to a combination of an HER2/neu antibody with a heme that form oligomers.

In a particular embodiment, invention relates to a combination of an HER2/neu antibody with a heme and/or a CAR combined with a heme that form oligomers.

In particular embodiment, the invention relates to a combination of Trastuzumab with a heme that form oligomers.

As used herein the term “oligomers” denotes stable complexes formed by the HER2/neu antibody and the heme and/or stable complexes formed by the CAR and the heme and correspond but are not limited to dimers, trimers, tetramers, pentamers or hexamers wherein heme particularly binds to the Ab on the heavy chain variable region and especially to the Fab portion.

A third aspect of the invention relates to a combination of an HER2/neu antibody with a heme and/or oligomers for medical use. In a particular embodiment, the invention relates to a combination of an HER2/neu antibody with a heme and/or CAR combined with a heme and/or oligomers for medical use.

In a particular embodiment, the invention relates to a combination of an HER2/neu antibody with a heme and/or oligomers for use in the treatment of a cancer.

In a particular embodiment, the invention relates to a combination of an HER2/neu antibody with a heme and/or chimeric antigen receptor and/or oligomers for medical use.

In another embodiment the invention relates to a combination of an HER2/neu antibody with a heme and/or oligomers for use in the treatment of a cancer in a subject in need thereof presenting an HER-2 overexpression.

In a particular embodiment the invention relates to a combination of an HER2/neu antibody with a heme and/or chimeric antigen receptor and/or oligomers for use in the treatment of a cancer in a subject in need thereof presenting an HER-2 overexpression.

In another embodiment the invention relates to an i) HER2/neu antibody and ii) a heme, as a combined preparation for simultaneous, separate or sequential use in a subject presenting an HER-2 overexpression.

In a particular embodiment the invention relates to an i) HER2/neu antibody and/or a CAR and ii) a heme, as a combined preparation for simultaneous, separate or sequential use in a subject presenting an HER-2 overexpression.

HER-2 overexpression can be detected by immunohistochemistry (IHC) or gene amplification analysed by fluorescence in situ hybridization (FISH). Particularly, methods and thresholds to determine HER-2 overexpression cancers are explained in “Recommendations for Human Epidermal Growth Factor Receptor 2 Testing in Breast Cancer: American Society of Clinical Oncology/College of American Pathologists Clinical Practice Guideline Update” written by Antonio C. Wolff in 2013.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “cancer” has its general meaning in the art and includes, but is not limited to, solid tumors and blood-borne tumors The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestinal, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant;

paraganglioma, malignant; extra-mammary paraganglioma, malignant; pheochromocytoma; glomangiosarcoma; malignant melanoma; amelanotic melanoma; superficial spreading melanoma; malign melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In a particular embodiment the cancer is a HER2/neu overexpressing cancer.

In a particular embodiment the term “HER2/neu overexpressing cancer” may be selected in the group consisting of breast cancers, cervical cancers, cholangiocarcinomas, extrahepatic colorectal cancers, intrahepatic colorectal cancers, esophageal and esophagogastric junction cancers, gallbladder cancer, gastric adenocarcinomas, head and neck carcinomas, hepatocellular carcinomas, intestinal (small) malignancies, lung cancer (non-small cells), melanomas, ovarian (epithelial) cancers, ovarian (non-epithelial) cancers, pancreatic adenocarcinomas, prostate cancers, unknown primary cancers, uterine cancers, testicular cancers, salivary duct carcinomas, colon cancer or bladder cancer.

In particular embodiment, the cancer is breast cancer.

In a particular embodiment the cancer can be a secondary, relapsed, resistant or refractory. As shown on the example, the inventors demonstrate that formation of oligomers improve the cytoxic potential of Trastuzumab mediated by CDC.

Thus, the invention also relates to a combination of an HER2/neu antibody with a heme and/or to the CAR combined to the heme and/or oligomers to improve CDC activity for use in the treatment of a cancer.

As used herein, the term “subject” denotes a mammal, such as a rodent, a feline, a canine, and a primate. Particularly, the subject according to the invention is a human.

In specific embodiments, it is contemplated that antibodies, CAR and oligomers of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters. A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Another modification of the antibodies, CAR and oligomers that is contemplated by the invention is a conjugate or a protein fusion of at least the antigen-binding region of the HER2/neu antibody with a heme and/or CAR combined with a heme and/or oligomers of the invention to serum protein, such as human serum albumin or a fragment thereof to increase half-life of the resulting molecule. Such approach is for example described in Ballance et al. EP0322094. Another possibility is a fusion of at least the antigen-binding region of the antibody of the invention to proteins capable of binding to serum proteins, such human serum albumin to increase half-life of the resulting molecule. Such approach is for example described in Nygren et al., EP 0 486 525.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule.

In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a-amino and e-amino groups of lysine through stable urethane linkages. Such copolymers retain the desirable properties of PEG, while providing reactive pendent groups (the amino group of lysine) at strictly controlled and predetermined intervals along the polymer chain. The reactive pendent groups 5 can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomerular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Glycosylation modifications can also induce enhanced anti-inflammatory properties of the antibodies by addition of sialylated glycans. The addition of terminal sialic acid to the Fc glycan reduces FcyR binding and converts IgG antibodies to anti-inflammatory mediators through the acquisition of novel binding activities (see Robert M. Anthony et al., J Clin Immunol (2010) 30 (Suppl 1):S9-S14; Kai-Ting C et al., Antibodies 2013, 2, 392-414).

### Therapeutic Composition

A fourth aspect of the invention relates to a method for treating cancer comprising administering to a subject in need thereof a therapeutically effective amount of a combination of an HER2/neu antibody with a heme and/or a CAR combined with a heme and/or of oligomers.

As used herein, the term “therapeutically effective amount” or “effective amount” refers to an amount effective, at dosages and for periods of time necessary, to achieve a desired therapeutic result. A therapeutically effective amount of the combination of an HER2/neu antibody with a heme and/or a CAR combined with a heme and/or the oligomers of the present invention may vary according to factors such as the disease state, age, sex, and weight of the individual, and the ability of the antibody of the present invention to elicit a desired response in the individual. A therapeutically effective amount is also one in which any toxic or detrimental effects of the combination of an HER2/neu antibody with a heme and/or a CAR combined with a heme and/or the oligomers are outweighed by the therapeutically beneficial effects. The efficient dosages and dosage regimens for the combination of an HER2/neu antibody with a heme and/or a CAR with a heme and/or the oligomers of the present invention depend on the disease or condition to be treated and may be determined by the persons skilled in the art. A physician having ordinary skill in the art may readily determine and prescribe the effective amount of the pharmaceutical composition required. For example, the physician could start doses of the oligomers of the present invention employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. Typically, the ability of a compound to inhibit cancer may, for example, be evaluated in an animal model system predictive of efficacy in human tumors. Alternatively, this property of a composition may be evaluated by examining the ability of the compound to induce cytotoxicity by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of an antibody of the present invention is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. In some embodiments, the efficacy may be monitored by visualization of the disease area, or by other diagnostic methods described further herein, e.g. by performing one or more PET-CT scans, for example using a labelled antibody of the present invention, fragment or mini-antibody derived from the antibody of the present invention. If desired, an effective daily dose of a pharmaceutical composition may be administered as two, three, four, five, six or more sub-doses administered separately at appropriate intervals throughout the day, optionally, in unit dosage forms. In some embodiments, the oligomers of the present invention are administered by slow continuous infusion over a long period, such as more than 24 hours, in order to minimize any unwanted side effects. An effective dose of an antibody of the present invention may also be administered using a weekly, biweekly or triweekly dosing period. The dosing period may be restricted to, e.g., 8 weeks, 12 weeks or until clinical progression has been established. As non-limiting examples, treatment according to the present invention may be provided as a daily dosage of an antibody of the present invention in an amount of about 0.1-100 mg/kg, such as0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

The antibodies, the hemes, the CAR and/or the oligomers of the invention formed by the combination of an HER2/neu antibody with a heme or by the combination of a CAR with heme may be administrated before or after surgery.

The antibodies, the hemes, the CAR and/or the oligomers of the invention formed by the combination of an HER2/neu antibody with a heme may be used alone or in combination with any suitable agent.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

In addition, other pharmaceutically acceptable forms include, e.g. tablets or other solids for oral administration; time release capsules; and any other form currently can be used.

In each of the embodiments of the treatment methods described herein, the combination of an HER2/neu antibody with a heme, and/or the combination of a CAR with a heme and/or of oligomers is/are delivered in a manner consistent with conventional methodologies associated with management of the disease or disorder for which treatment is sought. In accordance with the disclosure herein, an effective amount of the antibody or antibody-drug conjugate is administered to a patient in need of such treatment for a time and under conditions sufficient to prevent or treat the disease or disorder.

The present invention is also provided for therapeutic applications where the combination of an HER2/neu antibody with a heme, and/or the combination of a CAR with a hemeand/or of oligomers of the present invention may be used in combination with at least one further therapeutic agent, e.g. for treating cancer. Such administration may be simultaneous, separate or sequential. For simultaneous administration the agents may be administered as one composition or as separate compositions, as appropriate. The further therapeutic agent is typically relevant for the disorder to be treated. Exemplary therapeutic agents include other anti-cancer antibodies, cytotoxic agents, chemotherapeutic agents, radiotherapeutics agents, anti-angiogenic agents, anti-cancer immunogens, cell cycle control/apoptosis regulating agents, hormonal regulating agents, and other agents described below.

In some embodiments, the combination of an HER2/neu antibody with a heme, and/or a combination of a CAR with a heme and/or the oligomers of the present invention is/are used in combination with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1 ; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

In some embodiments, the combination of an HER2/neu antibody with a heme, and/or a combination of a CAR with a heme and/or the oligomers of the present invention is/are used in combination with a targeted cancer therapy. Targeted cancer therapies are drugs or other substances that block the growth and spread of cancer by interfering with specific molecules (“molecular targets”) that are involved in the growth, progression, and spread of cancer.

Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signalling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo[3,4-f][1,6]naphthyridin-3(2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In some embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the combination of an HER2/neu antibody with a heme, and/or the combination of a CAR with a heme and/or the oligomers of the present invention is/are used in combination with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ). Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents. Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behavior and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Combination compositions and combination administration methods of the present invention may also involve “whole cell” and “adoptive” immunotherapy methods. For instance, such methods may comprise infusion or re-infusion of immune system cells (for instance tumor-infiltrating lymphocytes (TILs), such as CC2+ and/or CD8+ T cells (for instance T cells expanded with tumor-specific antigens and/or genetic enhancements), antibody-expressing B cells or other antibody-producing or -presenting cells, dendritic cells (e.g., dendritic cells cultured with a DC-expanding agent such as GM-CSF and/or Flt3-L, and/or tumor-associated antigen-loaded dendritic cells), anti-tumor NK cells, so-called hybrid cells, or combinations thereof. Cell lysates may also be useful in such methods and compositions. Cellular “vaccines” in clinical trials that may be useful in such aspects include Canvaxin™, APC-8015 (Dendreon), HSPPC-96 (Antigenics), and Melacine® cell lysates. Antigens shed from cancer cells, and mixtures thereof (see for instance Bystryn et al., Clinical Cancer Research Vol. 7, 1882-1887, July 2001), optionally admixed with adjuvants such as alum, may also be components in such methods and combination compositions.

In some embodiments, the combination of an HER2/neu antibody with a heme, and/or the combination of a CAR with a heme and/or the oligomers of the present invention is/are used in combination with radiotherapy. Radiotherapy may comprise radiation or associated administration of radiopharmaceuticals to a patient. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

As used herein, the term “radiation therapy” or “radiotherapy” has its general meaning in the art and refers the treatment of colorectal cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a colorectal cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

In some embodiments, the combination of an HER2/neu antibody with a heme, and/or a combination of a CAR with a heme and/or the oligomers of the present invention is/are used in combination with an antibody that is specific for a costimulatory molecule. Examples of antibodies that are specific for a costimulatory molecule include but are not limited to anti-CTLA4 antibodies (e.g. Ipilimumab), anti-PD1 antibodies, anti-PDLL antibodies, anti-TIMP3 antibodies, anti-LAG3 antibodies, anti-B7H3 antibodies, anti-B7H4 antibodies or anti-B7H6 antibodies.

In certain embodiments, the use of liposomes and/or nanoparticles is contemplated for the introduction of antibodies, hemes and/or oligomers into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

In another embodiment, the further therapeutic active agent can be a hematopoietic colony-stimulating factor. Suitable hematopoietic colony-stimulating factors include, but are not limited to, filgrastim, sargramostim, molgramostim and epoietin alpha.

In still another embodiment, the other therapeutic active agent can be an opioid or non-opioid analgesic agent. Suitable opioid analgesic agents include, but are not limited to, morphine, heroin, hydromorphone, hydrocodone, oxymorphone, oxycodone, metopon, apomorphine, nomioiphine, etoipbine, buprenorphine, mepeddine, lopermide, anileddine, ethoheptazine, piminidine, betaprodine, diphenoxylate, fentanil, sufentanil, alfentanil, remifentanil, levorphanol, dextromethorphan, phenazodne, pemazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the further therapeutic active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

In yet another embodiment, the further therapeutic active agent can be a checkpoint blockade cancer immunotherapy agent.

Typically, the checkpoint blockade cancer immunotherapy agent is an agent which blocks an immunosuppressive receptor expressed by activated T lymphocytes, such as cytotoxic T lymphocyte-associated protein 4 (CTLA4) and programmed cell death 1 (PDCD1, best known as PD-1), or by NK cells, like various members of the killer cell immunoglobulin-like receptor (KIR) family, or an agent which blocks the principal ligands of these receptors, such as PD-1 ligand CD274 (best known as PD-Ll or B7-H1).

Typically, the checkpoint blockade cancer immunotherapy agent is an antibody. In some embodiments, the checkpoint blockade cancer immunotherapy agent is an antibody selected from the group consisting of anti-CTLA4 antibodies, anti-PD1 antibodies, anti-PDL1 antibodies, anti-PDL2 antibodies, anti-TIM-3 antibodies, anti-LAG3 antibodies, anti-IDO1 antibodies, anti-TIGIT antibodies, anti-B7H3 antibodies, anti-B7H4 antibodies, anti-BTLA antibodies, and anti-B7H6 antibodies.

The invention will be further illustrated by the following figures and examples. However, these examples and figures should not be interpreted in any way as limiting the scope of the present invention.

### FIGURES

FIG. 1: Exposure of Trastuzumab to heme results in broadening of antigen-binding reactivity. As evident on the graphs the antibody demonstrated heme concentration-dependent binding to structurally unrelated protein antigens (Thyroglobulin, Factor IX, hemoglobin, myoglobin, cytrochrome c). The reactivity of Trastuzumab to insulin is not affected by heme.

FIG. 2: Self-binding potential of native and heme-exposed Trastuzumab. Evaluation of the binding of native and heme exposed Trastuzumab to immobilized Trastuzumab by ELISA.

FIG. 3: Functional activity of heme-exposed Trastuzumab. Direct cytotoxicity and complement-dependent cytotoxicity of native and heme-exposed Trastuzumab on human breast cancer cells MDA MB-231 cells (left) and SKBR3 (right). The survival of cancer cells pre-treated or not with Trastuzumab was evaluated by using WST-1 metabolic dye in absence and presence of complement. Each point represents percentage of dead cells from triplicate wells. Here is depicted a representative example of three independent experiments.

FIG. 4: Functional activity of heme-exposed Trastuzumab. Complement-dependent cytotoxicity of separated fractions of heme-exposed Trastuzumab on human breast cancer cell line SKBR3. The different fractions of heme-exposed Trastuzumab were separated by size-exclusion chromatography. Each point represents percentage of dead cells from triplicate wells. Here is depicted a representative example of three independent experiments. Statistical analyses were performed by using Mann-Whitney test, star indicate: **:p<0.01; ***p<0.001; ****p<0.0001. HM—high molecular weight.

FIG. 5: Deposition of C3 on the surface of breast cancer cells after incubation with native and heme-exposed Trastuzumab. The antibody at 1 mg/ml was first pre-treated with 14 μM heme; after the tumour cells at density of 1×10⁶ were incubated with 10 μ/ml of native and heme-treated antibody. As a source of complement 50% human serum was applied. Following washing of the cells the deposition of activation fragments of C3 component was detected by specific antibody and flow cytometer.

### EXAMPLE

### Material & Methods

### Immunoblot

Lysates of Bacillus anthracis or human umbilical vein endothelial cells (HUVEC) were loaded on a 4-12% gradient NuPAGE Novex electrophoresis gel (Invitrogen, Carlsbad, Calif.). After migration, proteins were transferred on nitrocellulose membranes (iBlot gel transfer stacks, Invitrogen) by using iBlot electrotransfer system (Invitrogen). Membranes were blocked overnight at 4° C. in TBS containing Tween 0.1% (TBS-T). Next, the membranes were mounted on Miniblot system (Immunetics, Cambridge, Mass.) and incubated with Trastuzumab (0.2 μM) pre-treated with increasing concentrations of heme (0.15-20 μM) and then incubated for 2 h, at 22° C. Membranes were washed with TBS-T for 1 h before being incubated for 1 h with an alkaline phosphatase conjugated goat anti-human IgG (Southern Biotech, Birmingham, Ala.). Membranes were then washed again for 1 h before revealed with ready-to-use NBT/BCIP substrate solution (KPL Systems, USA).

### ELISA

### 1) Evaluation of Reactivity of Therapeutic Abs to a Panel of Antigens After Heme Exposure

Ninety-six well microtiter polystyrene plates (NUNC Maxisorp, Roskilde, Denmark) were coated with various antigens—human insulin; human hemoglobin, porcine thyroglobulin; horse cytochrome C; horse myoglobin (all from Sigma-Aldrich, St. Louis, Mo.), and human factor IX (LFB, France), at 10 μg/mL for 2 hours at room temperature. The plates were blocked with PBS containing 0.25% Tween 20 for 1 hour. Trastuzumab or Rituximab was treated at 2 μM in PBS with increasing concentrations of heme (0, 0.078-20 μM) for 5 minutes on ice. IgG was then diluted ten fold (0.2 μM final concentration) in PBS containing 0.05% Tween 20 (PBS-T) and incubated with immobilized antigens for 2 h at room temperature. After washing with PBS-T the plates were incubated for 1 hour with peroxidase-conjugated mouse anti-human IgG (clone JDC-10, Southern Biotech). Immunoreactivities were revealed using the o-phenylenediamine substrate (Sigma-Aldrich).

### 2) Evaluation of Induction of Antibody Homophilicity by Heme

Ninety-six well microtiter polystyrene plates (NUNC) were coated with Trastuzumab at 10 μg/mL for 2 hours at room temperature. The plates were blocked with PBS 0.25% containing Tween 20 for 1 hour. Trastuzumab that was biotinylated using EZ-Link™ NHS-LC-Biotin (ThermoFisher Scientific) was treated at 6.7 μM in PBS with 13.7 μM of heme (hemin, Sigma-Aldrich) for 5 minutes on ice. IgG was then diluted two times in PBS-T before incubation on plates for 2 h at room temperature. Following extensive washing, the plates were incubated for 30 min with streptavidin-HRP (Southern Biotech). After extensive washing with PBS-T, the immunoreactivities were revealed using the o-phenylenediamine substrate (Sigma-Aldrich).

For the evaluation of induction of antibody homophilicity by heme in low ionic strength conditions, same ELISA was performed using low salts concentration buffer (NaCl 15 mM) for the treatment of Trastuzumab with heme. To evaluate the specificity of heme, this method was performed using an analog of heme, zinc protoporphyrin (ZnPP). Trastuzumab was incubated at 6.7 μM in PBS with increasing concentrations of heme or ZnPP (0-64 μM). IgG was then diluted ten times in PBS-T before incubation on the plates for 2 h at room temperature.

### Protein Microarray Analyses

The bindings of Trastuzumab pre-incubated or not with heme were tested against more than 9000 human proteins (ProtoArray Human Protein Microarray v5.0, ThermoFisher Scientific, USA) using antibody specificity biomarker profiling protocol. First, the arrays were equilibrated at 4° C. for 15 min and then incubated with blocking buffer recommended by the manufacturer for 1 h at 4° C. on a circular shaker. After incubation, microarrays were washed once with PBS, 0.1% Tween 20, containing synthetic block (ThermoFisher Scientific) for 5 min. The monoclonal IgG1 (10 μM) was pre-treated or not with heme (20 μM). Following treatment Trastuzumab was further diluted to 33 nM (5 μg/ml) and added in the chamber containing the arrays. Following 90 min of incubation at 4° C. on a circular shaker, each array was washed 5 times for 5 min each at 4° C. To detect Trastuzumab, an Alexa Fluor 647 goat anti-human IgG antibody (ThermoFisher Scientific) was added to the incubation tray at 1 μg/ml for 90 min at 4° C. on a circular shaker. As described previously, the arrays were washed 5 times before being centrifuged in a 50 ml tube at 200×g for 1 minute at room temperature. Scanning of the arrays was performed on the next day with a GenePix 4000B Microarray Scanner. Fluorescence data were acquired by aligning the Genepix Array List onto the microarray using Genepix Pro analysis software. The resulting Genepix Results (GPR) files were imported into Invitrogen's Prospector 5.2 for further analysis.

### Real-Time Kinetic Analyses

### 1) Evaluation of the Binding of Heme to Trastuzumab

The binding kinetics and thermodynamics of interaction of Trastuzumab with heme was determined by surface plasmon resonance-based technique (BIAcore 2000, Biacore GE Healthcare, Sweden). Trastuzumab was immobilized on a CMS sensor chip (Biacore) by using amine-coupling kit provided by the manufacturer. The Ab was diluted in 5 mM maleic acid (pH 4) to a final concentration of 10 μg/ml. The achieved immobilization level was 6300 resonance unit (RU). All measurements were performed using HBS-EP (0.01M HEPES, pH 7.4 containing 0.15 M NaCl, 3 mM EDTA and 0.05% Tween 20). Initially, a stock solution of heme (hemin, Frontiers Scientific, Logan, Utah) at 1 mM was prepared in 0.05 N NaOH. Heme was further diluted to 10 μM in the running buffer and 8 two-fold dilutions (10-0.078 μM) were injected at flow rate of 30 μl/min. The association and dissociation phases of the interaction were monitored for 5 min and 10 min, respectively. The binding to the surface of the reference flow cell was subtracted from the binding to the Ab-coated flow cells. The regeneration of the bound-heme was achieved by exposure of the sensor surface to 300 mM imidazole. All binding analyses were performed at temperatures of 5, 10, 15, 20, 25, 30 and 35° C. The BIAevaluation version 4.1 software (Biacore) was used for the estimation of the kinetic rate constants. Calculations were performed by global analyses of the experimental data using the Langmuir binding with drifting base-line model included in the software.

To evaluate which portion of the Ab was binding to heme, Fab fragments, Fc fragments and the intact Trastuzuman were immobilized on a CMS sensor chip as described above. The achieved immobilization levels were 1500 RU for Fab fragments, 1450 RU for Fc fragments and 4300 RU for the intact Trastuzumab. Heme was diluted at 10 μM in HBS-EP and injected at flow rate of 30 ul/min. The association and dissociation phases of the interaction were monitored for 5 min and 10 min, respectively. Analysis was performed as described above.

### 2) Evaluation of the Interaction of Heme-Exposed Trastuzumab to Native Trastuzumab

Trastuzumab was immobilized on a CMS sensor chip (Biacore) by using amine-coupling kit (Biacore) after dilution in 5 mM maleic acid (pH 4) to a final concentration of 10 μg/ml. The achieved immobilization level was 5600 RU. All experiments were performed using HBS-EP. Trastuzumab was diluted to 10 μM in PBS and treated with 20 μM heme. After five minutes incubation on ice, two-fold dilutions of heme-exposed Trastuzumab (1000-1.95 nM) were injected at flow rate of 30 μl/min. The association and dissociation phases of the interaction were monitored for 4 min and 5 min, respectively. The binding to the surface of the reference flow cell was subtracted from the binding to the proteins-coated flow cells. The regeneration of the bound-IgG was achieved by exposure of the sensor surface to 150 mM imidazole. The real-time interaction analyses were performed at 10, 15, 25 and 35° C. The BIAevaluation version 4.1 software (Biacore) was used for the estimation of the kinetic rate constants. Calculations were performed by global analysis of the experimental data using the Langmuir binding with drifting base-line model included in the software.

### 3) Evaluation of the Binding of Heme-Exposed Trastuzumab to HER-2

Biotinylated HER-2 mimotope peptide [24] was immobilized on streptavidine (SA) sensor chip (Biacore) to a final concentration of 10 μg/ml. The achieved immobilization level was 500 RU. Trastuzumab (6.7 μM) was pre-treated with 13.404 heme. Native and heme-exposed Trastuzumab were diluted to 10 nM in the running buffer and 8 two-fold dilutions (10-0.078 nM) were injected at flow rate of 30 μl/min. The association and dissociation phases of the interaction were monitored for 5 min and 4 min, respectively. The binding to the surface of the reference flow cell was subtracted from the binding to the proteins-coated flow cells. The regeneration of the bound-Trastuzumab was achieved by exposure of the sensor surface to 1.5 M MgCl2. The BlAevaluation version 4.1 software (Biacore) was used for the estimation of the kinetic rate constants. Calculations were performed by global analysis of the experimental data using the Langmuir binding with drifting base-line model included in the software.

### 4) Evaluation of the Binding of Heme-Exposed Trastuzumab to FcRn

Recombinant human FcRn (kindly provided by Dr Sune Justesen, University of Copenhagen, Denmark) conjugated with biotin was immobilized on a straptavidin sensor chip (SA chip, Biacore) at a density of 1500 RU. Fc-γ fragments from Trastuzumab were generated by papain digestion. Native Fc-γ or Fc-γ treated in PBS at 12 μM with equimolar concentration of heme were diluted serially (two-fold each step) from 50 to 0.39 nM in 100 mM Tris-Citrate buffer pH 5.4, containing 0.1% Tween 20. The association and dissociation phases of the interaction were monitored for 4 min and 5 min, respectively. The sensor chip surfaces were regenerated by exposure to 100 mM Tris-Citrate buffer pH 7.4, containing 0.1% Tween 20 for 60 sec. All kinetic measurements were performed at temperature of 25° C. The evaluation of the kinetic data was performed by BIAevaluation version 4.1.1 Software (Biacore).

### Thermodynamic Analyses

For evaluation of the activation thermodynamic parameters of the interactions between heme and Trastuzumab, as well as the interaction of heme-exposed Trastuzumab to native Trastuzumab, the Eyring's approach was applied. The kinetic rate constants obtained at different temperatures were used to build Arrhenius plots. The values of slopes of the Arrhenius plots were calculated by using a linear regression analysis of the experimental kinetic data and substituted in Equations 1-4,

Ea=−slope×R (Eq. 1)

Where the “slope”=∂1n(ka/d/∂(1/T), and where Ea is the activation energy. The enthalpy, entropy, and Gibbs free energy changes characterizing the association phase were calculated using Equations 2-4,

ΔH=Ea−RT (Eq. 2)

1n(ka/d/T)=ΔH/RT+ΔS/R+1n(k′/h) (Eq. 3)

ΔG=ΔH−TΔS (Eq. 4)

where T is the temperature in Kelvin, k′ is the Boltzmann constant, and h is Planck's constant. All activation thermodynamic parameters were determined at the reference temperature of 25° C. (298.15 K).

To evaluate the changes of the thermodynamic parameters at equilibrium following equation were applied—

[symbol]

Geq= [symbol] G‡a− [symbol] G‡d,

[symbol]

Heq= [symbol] H‡a− [symbol] H‡d,

T [symbol] Seq=T [symbol] S‡a−T [symbol] S‡d

### Size-Exclusion Chromatography

Molecular composition of the native and heme-exposed Trastuzumab was compared by using FPLC Akta Purifier (GE, Healthcare), equipped with Superose 6 10/300 column. IgG was diluted to 6.7 μM in PBS and exposed to 13.4 μM of heme or heme analogues. In another experiment, Trastuzumab (6.7 μM) was pre-treated with potassium cyanide (KCN, 10 mM final concentration) before treatment with heme (13.4 μM). One ml of each native or heme-exposed Ab was loaded on column equilibrated with the corresponding buffer. The flow rate of 0.5 ml/min was used. Chromatograms were recorded by using UV detection of protein at wavelength of 280 nm and at 400 nm for heme detection. The obtained fractions were collected separately in order to test their individual therapeutic effect subsequently.

### Transmission Electron Microscopy

Trastuzumab was dialyzed to HBS buffer and diluted to 1 mg/ml (6.7 μM). Heme (1 mM stock in 0.05 N NaOH) was added to the Ab solution to final concentration of 13.4 μM. Different concentrations of native and heme-exposed Trastuzumab were first assessed—150 μg/ml; 30 μg/ml and 7.5 μg/ml. Six microliters of each sample are placed on a carbon-copper grid (300 mesh) for 1 min at room temperature after a standard glow discharge procedure (2 mA, 0.3 mBar, 40 sec). After adsorption, the excess is removed by blotting using a Whatman grade 5 paper. Grids are then stained with uranyl acetate 2%, one drop quickly and the second one for 1 minute at room temperature. They are finally blotted with Whatman grade 5 paper and air-dried. Specimen are then observed under a 200 kV F20 (FEI) transmission electron microscope and acquisition of the images is carried out using a 2 k×2 k USC1000 camera (GATAN). All observations are made at magnifications ×50 000 and ×62 000.

### Absorbance Spectroscopy

Absorbance spectra were measured by using UNICAM Helios b, UV-vis spectrophotometer. Trastuzumab was diluted to 2 μM in PBS and titrated with increasing concentrations of heme (0.125-64 μM). Aliquots of heme stock solution (1 mM in 0.05 M NaOH) were added both to cuvette containing Trastuzumab and to a reference cuvette, containing PBS only. After addition of each heme aliquot and incubation for 2 min in dark, the absorbance spectra in the wavelength range 350-700 nm were recorded. The spectra were scanned at rate of 300 nm/min. All measurements were performed at room temperature, in quartz cuvettes with optical path of 1 cm.

### Fluorescence Spectroscopy

Quenching of intrinsic tryptophan fluorescence of Trastuzumab by heme was measured by using Hitachi F-2500 fluorescence spectrophotometer (Hitachi Instruments Inc., UK). The Ab was diluted to 0.1 μM in PBS and titrated with increasing concentrations (0, 0.01, 0.025, 0.05, 0.1, 0.2, 0.4, 0.8, 1.6 and 3.2 μM) of heme, added as aliquots from a stock solution. A wavelength of 295 nm was used to selectively excite tryptophan residues. Excitation and emission slits were both adjusted to 10 nm. The emission spectra of Trastuzumab was measured in the wavelength range 300-450 nm, at scan speed of 300 nm/min. Quartz cuvette with 1 cm optical path was used in the experiment. All measurements were performed at room temperature.

### Circular Dichroism

The CD spectra measurements were performed with JASCO-J710 Spectrometer. Data pitch and slit were both set to 1 nm. The data were recorded at scan speed on 10 nm/min in the range of 260-185 nm. Before the measurements Trastuzumab was dialyzed against 10 mM Na phosphate buffer pH 7.4 and diluted in the same buffer to a final concentration of 1 μM. The Ab was exposed or not to 10 μM concentration of heme. All spectra were acquired at 20° C. in quartz cuvettes with 1 mm optical path.

### Molecular Docking

Molecular docking studies were performed using the Autodock (version 4.2.6) software tool [25]. The X-ray crystallographic structure of Trastuzumab was acquired from the protein data bank (PDB) at a resolution of 2.08 Å (4 HKZ). Protein L and protein A fragments were removed from the file. The model was prepared by adding Gasteiger charges and optimizing torsion angles, and saved in PDBQT format. All water molecules were removed from the macromolecule and polar hydrogen atoms were added. A blind molecular docking method was used to dock heme on Fab fragment of Trastuzumab. A second docking was made on variable region of Trastuzumab for more precise analysis. The first grid was generated around the whole structure, the second one was calculated based on following coordinates (X=−16.139, Y=−17.886 and Z=9.161) in order to encompass the entire variable site. Lamarckian genetic algorithm (LGA) was selected for freezing, docking with default parameters in Autodock. The ten best conformations were selected and their energies calculated.

### Cell Lines

MDA-MB-231 cells were cultured in DMEM F-12 medium and supplemented with 10% heat-inactivated FBS, 1% Penicilin-Streptomycin. SKBR3 cells were culture in Mc Coy's 5A medium and supplemented with 10% heat-inactivated FBS, 1% Penicilin-Streptomycin. All the cells were maintained in an incubator at 37° C. with 5% CO2 and their viability was controlled by Trypan Blue.

### Flow Cytometry—Evaluation of Trastuzumab Binding to Breast Cancer Cells

MDA-MB-231 or SKBR3 cells were blocked in medium 10% FCS for 15 minutes and washed in PBS for 10 minutes. Cells were re-suspended in PBS and treatments were added. Trastuzumab diluted at 10 mg/ml in PBS was treated or not with 50 μM heme. Cells were incubated with a final concentration of Trastuzumab of 10 μg/ml. As controls, same resulting quantities of heme were added in separate tubes. Moreover, another IgG1 (Rituximab, Roche) was treated with heme at the same concentrations as a control. The cells were treated for 1 hour at 37° C. before washed two times in PBS. To detect the bound IgG, FITC-conjugated rabbit anti-human IgG (Southern Biotech) was incubated for 30 minutes at room temperature. Cells were washed once with PBS before analyses by using LSRII BD Flow Cytometer (BD Immunocytometry Systems, San Jose, Calif.). A total of 10 000 gated events were analyzed per sample.

### Direct and Complement-Mediated Cytotoxicity

Cells were treated on 96-wells plates with flat bottom and low evaporation lid (Coster, USA). MDA-MB-231 or SKBR3 breast cancer cells were plated and left for 2 hours to adhere. Trastuzumab diluted to 1 mg/ml was treated or not with 20 μM heme. The Ab was diluted to a final concentration of 3.7 μg/ml. As a source of complement, baby rabbit complement (AbD Serotec) was added to obtain a dilution of ×5 (MDA-MB-231 cells) or ×8 (SKBR3 cells). As control, heat inactivated fetal calf serum was added at the same ratios. To evaluate the amount of alive cells, WST-1 dye (Roche) was added after 5 days of incubation at 37° C. The absorbance at 450 nm and 690 nm was read after 2 hours incubation at 37° C.

### Antibody-Mediated Cellular Cytotoxicity

PBMCs were isolated from healthy donor blood (Établissement Français du Sang [EFS] Cabanel, Paris, France) by density gradient using Ficoll-Paque Plus (GE-Healthcare, UK), and used as a source of NK cells. PBMCs were seeded in a 24-wells plate and stimulated with IL-2 (100 UI/ml) overnight at 37° C. and 5% CO2 in RPMI 1640 medium with L-glutamine and 10% of heat-denatured FBS (Gibco, Life Technologies, USA). The next day, 5000 breast cancer cells/well prepared in FBS-free medium were transferred to a 96-well plate with round bottom. Trastuzumab (1 mg/ml) was pre-incubated or not with heme (20 μM) and was then diluted to 10 μg/ml before being added to the cancer cells. The plate was incubated for 30 min at 37° C. PBMCs were then mixed with cancers cells at different E:T ratios (from 60:1 to 3.75:1) and co-cultured for 4 h at 37° C. After the incubation, the cytotoxic effects of PBMCs on cancer cells were determined using the CytoTox 96 Non-Radioactive Cytotoxicity Assay kit (Promega).

### Results

### Heme Induces Antigen-Binding Polyreactivity of Trastuzumab

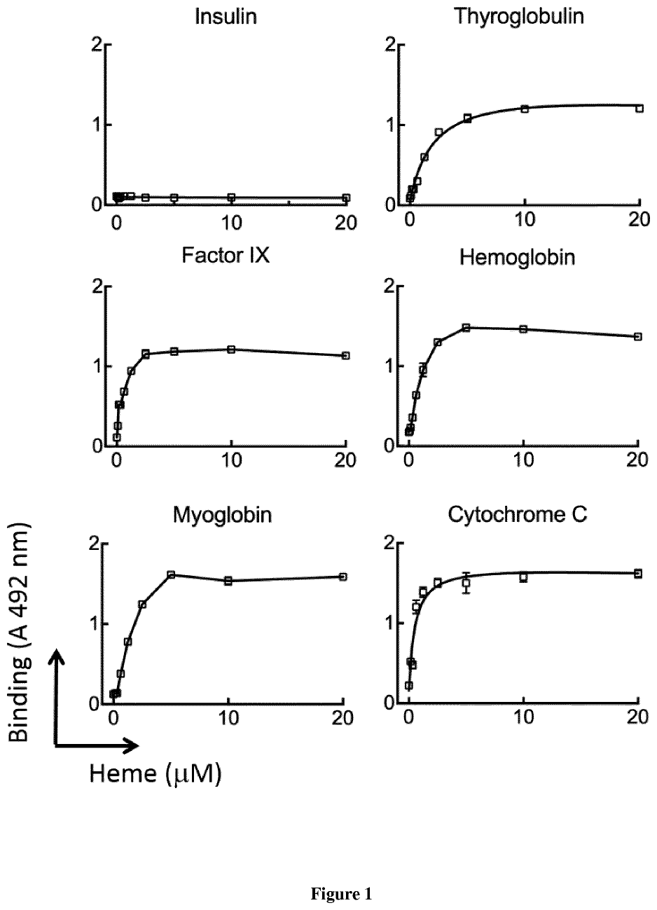

Previously, it was reported that Trastuzumab acquires reactivity towards protein and lipid autoantigens after heme exposure [19]. We first aimed to characterize the extent of the new binding specificity of heme-exposed Trastuzumab. To this end, we compared the reactivity of native and heme-exposed Trastuzumab to bacterial and endothelial antigens using immunoblot analyses (Data not shown). The results showed that exposure to heme results in a concentration-dependent increase of binding to the bacterial and autoantigens. These results were further supported by ELISA assay where reactivity of the Ab to a panel of unrelated polypeptide antigens was characterized. Heme-exposed Trastuzumab demonstrated binding to most of them, in a heme concentration dependent manner (FIG. 1). To investigate a broader spectrum of target recognition, we compared the reactivity of native and heme-exposed Trastuzumab to more than 9,000 human proteins using ProtoArray® technology. As expected, native Trastuzumab demonstrated a binding to very few proteins. Strikingly, heme-exposed Trastuzumab showed a considerable gain of reactivity by binding to a large number of human proteins (Data not shown). Quantitative analyses of the binding indicated that among the most strongly recognized targets of the heme-exposed Trastuzumab there were both intracellular and extracellular proteins (Data not shown). It is also important to note that heme alone was able to bind to numerous human proteins, but to a lower extent as compared to the antibody exposed to heme.

Next, we investigate the binding of the antibody to its cognate target, HER2/neu, upon heme exposure. The binding of heme-exposed Trastuzumab to HER2/neu was assessed by flow cytometry analyses using two different human breast cancer cell lines—SK-BR-3 and MDA-MB-231 (Data not shown). SK-BR-3 line has a high level of expression of HER2/neu, whereas MDA-MB-231 has a very low level of expression of HER2/neu. Accordingly, the binding of native Trastuzumab to MDA-MB-231 cells was characterized with a low intensity. No significant difference was observed in the binding of the heme-exposed Ab (Data not shown). The binding of Trastuzumab to high-HER2/neu expressing cell line showed also no difference between native and heme-exposed Ab (Data not shown). Finally, the binding of the native and heme-exposed Trastuzumab to its cognate target was determined by surface plasmon resonance (SPR)-based technique (Data not shown). HER2/neu mimotope was immobilized on a sensor chip and the binding of increasing concentrations of native or heme-exposed Trastuzumab was measured. Although heme-exposed Trastuzumab showed a lower binding response, both forms of the Ab demonstrated a considerable binding to HER2/neu mimotope, with binding affinities in the same order (KD values of 3.8 nM and 6.6 nM for the native and heme-treated Ab, respectively). To investigate whether heme exposure affects the Fc portion of Trastuzumab, the binding of heme-exposed Trastuzumab to recombinant human neonatal Fc receptor (FcRn) was determined by SPR-based technique. Estimation of the kinetic parameters showed that native and heme-exposed Trastuzumab bind to the receptor with identical binding affinities (Data not shown).

Taking together, these data demonstrated that Trastuzumab acquires polyreactivity upon heme exposure without affecting its ability to bind to HER2/neu and to interact with FcRn receptor.

In addition, the potential of other compounds, derivatives of heme, to induce polyreactivity of Trastuzumab was assessed. Thus, the exposure of Trastuzumab to Fe (III) mesoporphyrin IX, Fe (III) deuteroporphyrin IX and Fe (III) coproporphyrin I, resulted in an appearance of novel antigen-binding specificities (data not shown). In contrast free iron ions or protoporphyrin IX structure devoid of metal ion were not able to modify the specificity of Trastuzumab. These result clearly demonstrate that the most potent inducer of polyreactivity of Trastuzumab is Fe (III) deuteroporphyrin IX, whereas Fe (III) protoporphyrin IX has the lowest potential to uncover reactivity towards bacterial antigens (data not shown). These results indicate that Fe-containing porphyrin molecular system is indispensable for uncovering the polyreactivity of Trastuzumab.

### Heme Binds with a High Affinity to Variable Region of Trastuzumab

Our results indicated that exposure of Trastuzumab to heme induces a new pattern of antigen recognition. To understand the mechanism of these changes in the antigen binding function of the Ab, we investigated the interaction between heme and Trastuzumab. Absorbance spectroscopy revealed that the exposure of the Ab to heme resulted in an increased absorbance intensity of heme in high and low energy regions of the spectrum (Data not shown). These changes in the spectral characteristics of heme are consistent with a specific binding of the tetrapyrrole compound to the protein molecule. Besides the absorbance spectroscopy, the ability of heme to interact with Trastuzumab was investigated by using fluorescence spectroscopy. To this end, the quenching of the intrinsic tryptophan fluorescence of the Trastuzumab was measured as a function of the concentration of heme. Exposure to heme resulted in a concentration-dependent decrease in the fluorescence signal of the therapeutic Ab (Data not shown). Furthermore, the binding of heme to Trastuzumab was investigated by circular dichroism spectroscopy (Data not shown). Considerable changes of the circular dichroism ellipticity curves were observed after exposure of the Ab to heme. This result indicates that exposure to heme resulted in alteration in the secondary structure of the IgG. This data is in accordance with data from absorbance and fluorescence spectroscopy demonstrating that heme interacts and binds directly to the Ab.

The interaction of heme with Trastuzumab was further investigated by SPR (Data not shown). Heme bound to immobilized Trastuzumab with a high affinity (KD value of 100 nM). As can be deduced by the slow dissociation observed on real time binding profiled (Data not shown), Trastuzumab formed stable complexes with heme. The interaction between heme and Trastuzumab was further investigated at different temperatures. The association rate constant increased with an increase of the temperature. The dissociation rate constant was also sensitive to temperature. However, the augmentation of the temperature resulted in decrease in the dissociation rate i.e. in an increase in the stability of the intramolecular interaction. The temperature dependencies of the rate constants were further used to evaluate the thermodynamic parameters for the association, the dissociation, and the equilibrium of the interaction of heme with the monoclonal Ab. The change in the entropy during the association was with a negative value (TΔS=−12.4±7.3 kJ mol-l). The apparent value of the changes of enthalpy during association was positive (ΔH=40.2±7.2 kJ mol-l). During dissociation, the apparent values of ΔH and TΔS were both with negative values. At equilibrium heme binding to Trastuzumab was characterized with unfavorable change in the enthalpy (ΔH=87.7±18.9 kJ mol-l) and highly favorable changes in the binding entropy (TΔS=127±18.3 kJ mol-l). These data demonstrated that heme binding to the Ab is entropy-driven and enthalpy controlled process. Overall, the results from the thermodynamic analyses indicate that the binding of heme to Trastuzumab does not require major structural adaptations of the protein. The favorable entropy changes most probably arise from disruption of the solvatation shell of heme.

Further, we investigated the position of heme binding site in the IgG molecule. To this end, the SPR experiment was conducted using Fab fragments and Fc fragments of Trastuzumab. Heme demonstrated a preferential binding to the immobilized Fab portion of the Ab (Data not shown).

Finally, the heme-binding site on the Fab fragment of Trastuzumab was predicted by molecular docking using Autodock software (Data not shown). The first four most probable sites of heme binding, based on the binding energy score, on the variable region of Trastuzumab were found to be on the heavy chain variable region. The putative heme-binding site partly overlaps with the CDR H2 loop. Molecular docking analyses predicted that heme is bound to the polypeptide chain in such a way that it remains at large extend exposed to the solvent.

### Heme Induces Self-Association of Trastuzumab

To investigate whether heme binding affects the molecular composition of Trastuzumab we applied size exclusion chromatography. While native Trastuzumab eluted only as monomers, heme-exposed Trastuzumab eluted both as monomeric and oligomeric species. Moreover, it was observed that heme co-localized with the oligomeric forms of Trastuzumab (Data not shown). The effect of heme on the molecular composition of the Ab was concentration-dependent (Data not shown). Further, Fc and Fab fragments of Trastuzumab were exposed to heme and analyzed in the same conditions (Data not shown). Upon heme exposure, Fc fragments remained in a monomeric form. In contrast, heme-treated Fab fragments of Trastuzumab were eluted in two distinct molecular species—monomers and dimers. Heme co-localized with the dimeric species of Fab.

Next, to further characterize the mechanism of formation of the soluble oligomers of Trastuzumab, heme was pre-treated with cyanide before addition of the Ab (Data not shown). Cyanide anion is a high affinity ligand of heme's iron and as a consequence blocks metal's coordination potential and redox chemistry. The pre-treatment of heme with cyanide inhibited its potential to induce formation of soluble oligomers of the monoclonal Ab (Data not shown). To further substantiate this result, an additional experiment was performed using Zn (II) protoporphyrin IX (ZnPP) instead of heme (Data not shown). Treatment of Trastuzumab with ZnPP failed to induced antibody homophilicity or oligomerization. Taken together these results indicate that the iron in the tetrapyrrole structure of heme plays a crucial role in the formation of the soluble oligomers of Trastuzumab. A close structural analogue of heme—Fe(III)mesoporphyrin IX was also able to induce oligomerization of Trastuzumab. Proteoporphyrin IX (heme analogue devoid of Fe ion) or free iron ions were not able to induce self-association of the antibody (Data not shown). This result indicate that porphyrin molecular system that contains Fe(III) ion is indispensable for triggering of the self-association of Trastuzumab.

To investigate whether the formation of soluble oligomers induced by heme exposure is typical only for Trastuzumab, we analyzed molecular profiles of five additional therapeutic monoclonal Abs. No formation of oligomers was observed following exposure of these therapeutic Abs to heme (Data not shown).

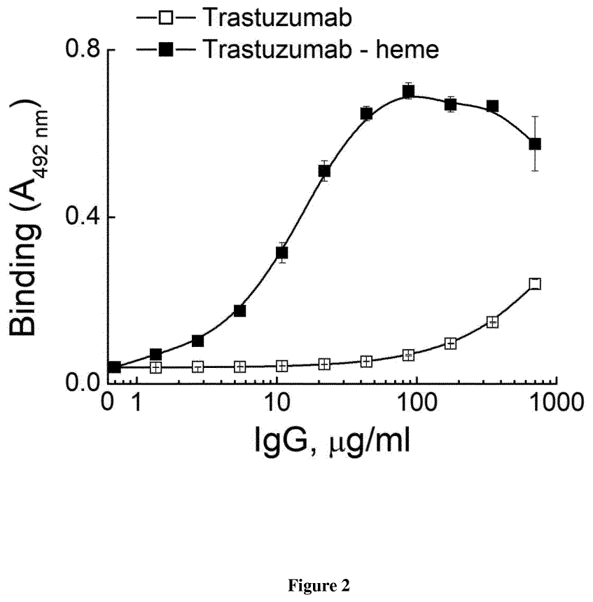

To acquire more details about the self-binding tendency of heme-exposed Trastuzumab, the induction of antibody homophilicity by heme was first evaluated by ELISA (FIG. 2). Biotinylated Trastuzumab was treated with heme and incubated on plates with immobilized native Trastuzumab. Whereas native Trastuzumab showed only negligible self-binding activity, the heme-treated Ab demonstrated a strong binding in a bell-shaped dependent manner, which is typical for interactions of homophilic Abs (FIG. 2). To further characterize the self-binding of Trastuzumab induced by heme, kinetic measurements were performed. The heme-treated Ab bound to itself in a dose-dependent manner (Data not shown). The kinetic analyses confirmed that Trastuzumab is able to bind to itself with physiological relevant affinity (KD value of 92.3 nM at 25° C.). Thermodynamic analyses of self-association of heme-bound Trastuzumab revealed that the process is entropy-driven and enthalpy-controlled (Data not shown). Noteworthy, similar thermodynamic mechanism was observed in the case of heme binding to the Ab (Data not shown). This result suggests that the main driver for self-association of Trastuzumab is heme.

To characterize the heme-mediated oligomerization of Trastuzumab, a negative stain transmission electron microscopy technique was used. The morphologies of native and heme-bound Trastuzumab were compared (Data not shown). The visualization of native Ab showed a typical appearance of objects containing three globular domains, as expected for an intact IgG molecule (Data not shown). In the case of heme-exposed Trastuzumab, in addition to monomeric structures there were molecular species containing two or three IgG molecules (Data not shown). Importantly, self-binding of Trastuzumab molecules resulted in well-organized species but not in a random aggregation. Next, we used protein A bound to colloidal gold to specifically label Fc fragments of Trastuzumab (Data not shown). This labelling allowed us to confirm that Fc fragments were not involved in the interactions between heme-bound Trastuzumab. In summary these results indicate that formation of supramolecular species of the Ab is due to interactions between the variable regions.

### Heme Exposure Increases Tumor Killing Potential of Trastuzumab

To provide understanding about functional impact of heme binding to the therapeutic Ab, we tested its ability to kill malignant cells. Several mechanisms of action of Trastuzumab have been described and discussed in the literature. The main ones include direct action on cancer cells by blockage of the receptor and antibody-dependent cellular cytotoxicity (ADCC) [21,22,23].

First, the antibody-dependent cellular cytotoxicity of native and heme exposed Trastuzumab was investigated (Data not shown). In this experiment, the breast cancer cell lines were incubated with the native or heme-exposed Trastuzumab and then incubated in presence of freshly isolated PBMCs. After 4 hours of incubation, the cell lysis was quantified by the release of lactate dehydrogenase. No difference in the cytotoxic potential was observed between the native and heme-exposed Trastuzumab on the HER2/neu low expressing and HER2/neu high expressing cell lines (Data not shown).

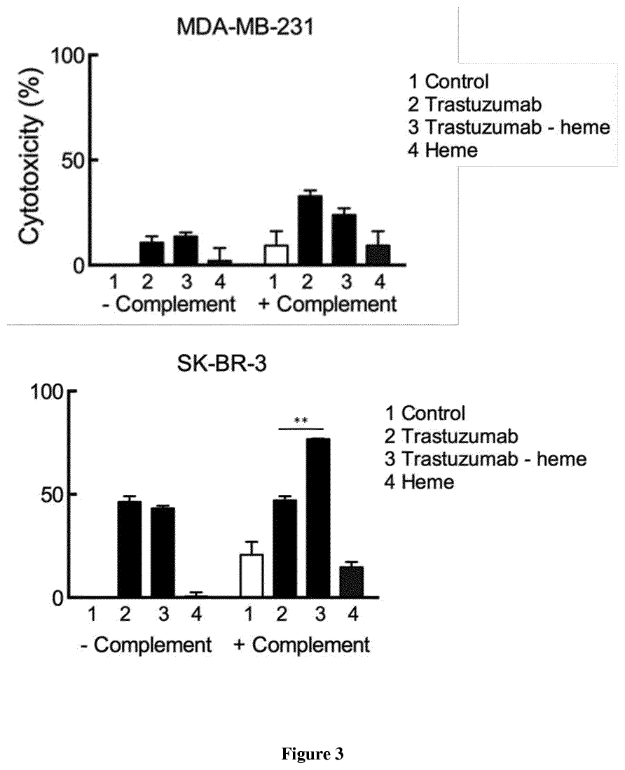

Next, we determined if the direct cytotoxicity and complement-dependent cytotoxicity (CDC) of Trastuzumab were affected by interaction with heme. SK-BR-3 and MDA-MB-231 cells were treated with native Trastuzumab or heme-exposed Trastuzumab in the presence—or not of complement. The SK-BR-3 cells showed a clear cytotoxicity in the presence of Trastuzumab. Interestingly, in the presence of complement, the cytotoxicity was significantly higher in the case of heme-bound Trastuzumab than the native form (p <0.01, Mann-Whitney test; FIG. 3). When we assessed the cytotoxicity of low HER2/neu expressing cancer cell line—MDA-MB-231, in the presence of complement a negligible decrease in the percentage of live cells was observed, but no significant difference was detected between the native and the heme-exposed Ab.

To exclude a potential cytotoxic action of heme on the cells, both cell lines were also treated with heme alone at identical concentrations as those introduced by heme-bound Trastuzumab. This treatment has no negative impact on the cell proliferation.

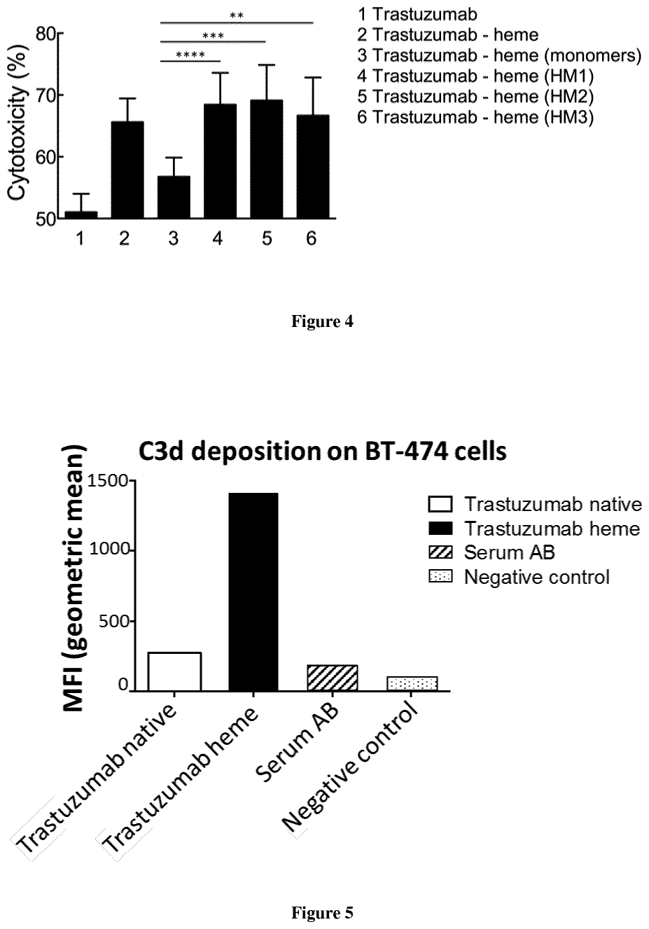

Next, we assessed whether the cytotoxic action of heme-bound Trastuzumab on the SK-BR-3 cells was due to the oligomers induced by heme binding (FIG. 4). To this end, Trastuzumab was treated with heme, and the 3 oligomeric fractions as well as the monomeric one were collected separately by a size-exclusion chromatography. In the same experiment settings as described above, the SK-BR-3 cells were treated with the different molecular species in the presence of complement. The monomeric fraction demonstrated the same cytotoxic effect as the native Trastuzumab. Interestingly, the oligomeric fractions showed the same cytotoxic effect as the heme-treated Trastuzumab, which were significantly different from the monomeric fraction (FIG. 4). Moreover, the first two oligomeric fractions demonstrated an even higher cytotoxic effect than the heme-treated Ab. This result demonstrates that the increased cytotoxic potential of heme-exposed Trastuzumab on the HER2/neu-positive cells is mediated by the dimers and higher molecular species of the IgG.

Moreover, deposition of C3 on the surface of breast cancer cells after incubation with native and heme-exposed Trastuzumab was assessed. This experiment clearly demonstrate that heme-exposed Trastuzumab has considerably high capacity to activate the complement on the cellular surface and to induce opsonisation of the cancer cells with C3. Thus, the heme-exposed Trastzumab can facilitate cell elimination through phagocytosis.

### REFERENCES

Throughout this application, various references describe the state of the art to which this invention pertains. The disclosures of these references are hereby incorporated by reference into the present disclosure.

https://www.ncbi.nlm.nih.gov/pmc/articles/PMC3896214/

https://www.ncbi.nlm.nih.gov/pubmed/16458110

https://www.ncbi.nlm.nih.gov/pubmed/25716732

1 Watson, C. D. & Ford, H. C. High-affinity binding of riboflavin and FAD by immunoglobulins from normal human serum. Biochem. Int. 16, 1067-1074 (1988).

2 Stoppini, M. et al. Characterization of the two unique human anti-flavin monoclonal immunoglobulins. Eur. J Biochem. 228, 886-893 (1995).

3 Zhu, X., Wentworth, P., Jr., Kyle, R. A., Lerner, R. A. & Wilson, I. A. Cofactor683 containing antibodies: crystal structure of the original yellow antibody. Proc. Natl. Acad. Sci. USA. 103, 3581-3585 (2006).

4 Rajagopalan, K. et al. Novel unconventional binding site in the variable region of immunoglobulins. Proc. Natl. Acad. Sci. USA. 93, 6019-6024 (1996).

5 Dimitrov, J. D. et al. Antibodies use heme as a cofactor to extend their pathogen elimination activity and to acquire new effector functions. J. Biol. Chem. 282, 26696-26706 (2007).

6 McIntyre, J. A. The appearance and disappearance of antiphospholipid autoantibodies subsequent to oxidation--reduction reactions. Thromb. Res. 114, 579-587 (2004).

7 McIntyre, J. A., Wagenknecht, D. R. & Faulk, W. P. Autoantibodies unmasked by redox reactions. J. Autoimmun. 24, 311-317, (2005).

8 McIntyre, J. A. & Faulk, W. P. Redox-reactive autoantibodies: biochemistry, characterization, and specificities. Clin. Rev. Allergy Immunol. 37, 49-54, (2009).

9 Dimitrov, J. D. et al. A cryptic polyreactive antibody recognizes distinct clades of HIV-1 glycoprotein 120 by an identical binding mechanism. J. Biol. Chem. 289, 17767-17779, (2014).

10 Lecerf, M. et al. Prevalence and gene characteristics of antibodies with cofactor induced HIV-1 specificity. J. Biol. Chem. 290, 5203-5213, d (2015).

11 Hadzhieva, M. et al. Mechanism and Functional Implications of the Heme-Induced Binding Promiscuity of IgE. Biochemistry 54, 2061-2072, (2015).

12 Pavlovic, S. et al. Intravenous immunoglobulins exposed to heme (heme IVIG) are more efficient than IVIG in attenuating autoimmune diabetes. Clin. Immunol. 138,162-171, (2011).

13 Gupta, N. et al. Neutralization of Japanese Encephalitis Virus by heme-induced broadly reactive human monoclonal antibody. Sci. Rep. 5, 16248, (2015).

14 Djoumerska-Alexieva, I., Roumenina, L. T., Stefanova, T., Vassilev, T. & Dimitrov, J. D. Heme-Exposed Pooled Therapeutic IgG Improves Endotoxemia Survival. Inflammation 40, 117-122, (2017).

15 Dutra, F. F. & Bozza, M. T. Heme on innate immunity and inflammation. Front. Pharmacol. 5, 115, (2014).

16 Soares, M. P. & Bozza, M. T. Red alert: labile heme is an alarmin. Curr. Opinion Immunol. 38, 94-100, d (2016).

17 Roumenina, L. T., Rayes, J., Lacroix-Desmazes, S. & Dimitrov, J. D. Heme: Modulator of Plasma Systems in Hemolytic Diseases. Trends Mol. Med. 22, 200-213, (2016).

18 Leavy, O. Therapeutic antibodies: past, present and future. Nat. Rev. Immunol. 10, 297, (2010).

19 McIntyre, J. A. & Faulk, W. P. Autoantibody potential of cancer therapeutic monoclonal antibodies. Intl. J. Cancer (2009).

20 Krysko, D. V. et al. Immunogenic cell death and DAMPs in cancer therapy. Nat. Rev. Cancer 12, 860-875, doi:10.1038/nrc3380 (2012).

21 Hudis, C. A. Trastuzumab—mechanism 724 of action and use in clinical practice. New Engl. J. Med. 357, 39-51, doi:10.1056/NEJMra043186 (2007).

22 Spector, N. L. & Blackwell, K. L. Understanding the mechanisms behind trastuzumab therapy for human epidermal growth factor receptor 2-positive breast cancer. J. Clin. Oncol. 27, 5838-5847, (2009).

23 Vu, T. & Claret, F. X. Trastuzumab: updated mechanisms of action and resistance in breast cancer. Front. Oncol. 2, 62, (2012).

24 Shang, Y., Singh, P. R., Chisti, M. M., Mernaugh, R. & Zeng, X. Immobilization of a human epidermal growth factor receptor 2 mimotope-derived synthetic peptide on Au and its potential application for detection of herceptin in human serum by quartz crystal microbalance. Anal. Chem. 83, 8928-8936, (2011).

25 Morris, G. M. et al. AutoDock4 and AutoDockTools4: Automated docking with selective receptor flexibility. J. Comput. Chem. 30, 2785-2791, (2009).

1. A combination of an HER2/neu antibody with a heme.

2. The combination according to claim 1 wherein the heme is selected from the group consisting of heme a, heme c, heme d, heme d₁, heme o, heme P460, siroheme, and a Fe porphyrine.

3. The combination according to claim 1 wherein the HER2/neu antibody is Transtuzumab.

4. The combination according to claim 1 wherein the HER2/neu antibody and heme form oligomers.

5. A chimeric antigen receptor which comprises at least one VH and/or VL sequence of the HER2/neu antibody combined with a heme.

6. The chimeric antigen receptor of claim 5 which further comprises an extracellular hinge domain, a transmembrane domain, and an intracellular T cell signaling domain.

7. The chimeric antigen receptor of claim 5 comprising an antigen-binding domain comprising a single chain variable fragment (scFv) of the HER2/neu antibody.

8. (canceled)

9. (canceled)

10. The method of claim 14, wherein the combination or the chimeric antigen receptor is administered with at least one additional anti-cancer agent.

11. The method of claim 14, wherein the cancer is selected from the group consisting of breast cancers, cervical cancers, cholangiocarcinomas, extrahepatic colorectal cancers, intrahepatic colorectal cancers, esophageal and esophagogastric junction cancers, gallbladder cancer, gastric adenocarcinomas, head and neck carcinomas, hepatocellular carcinomas, intestinal (small) malignancies, lung cancer (non-small cells), melanomas, ovarian (epithelial) cancers, ovarian (non-epithelial) cancers, pancreatic adenocarcinomas, prostate cancers, unknown primary cancers, uterine cancers, testicular cancers, salivary duct carcinomas, colon cancer and bladder cancer.

12. A therapeutic composition comprising an HER2/neu antibody, a heme and/or a chimeric antigen receptor and at least one excipient.

13. (canceled)

14. A method for treating cancer comprising administering to a subject in need thereof a therapeutically effective amount of
a combination of a HER2/neu antibody and a heme and/or
a chimeric antigen receptor which comprises at least one VH and/or VL sequence of an HER2/neu antibody combined with a heme.

15. The combination according to claim 2 wherein the Fe porphyrine is Fe (III) mesoporphyrin IX, Fe (III) protoporphyrin IX, Fe (III) deuteroporphyrin IX, Fe (III) hematoporphyrin IX, or Fe(III) coproporphyrin I.
